(19) United States
(12) Patent Application Publication
(10) Pub. No.: US 2016/0135721 A1
(43) Pub. Date: May 19, 2016

(54) Catheter Comprising a Detection Device for Supplying Real-Time Detection of a Sample Material

(71) Applicant: GILUPI GMBH, Potsdam (DE)

(72) Inventors: Andreas Bollmann, Berlin (DE); Robert Niestroj-Pahl, Potsdam (DE); Klaus Lücke, Potsdam (DE); Solveigh Krusekopf, Berlin (DE)

(21) Appl. No.: 14/899,441

(22) PCT Filed: Jun. 23, 2014
(86) PCT No.: PCT/EP2014/063165

§ 371 Date: Dec. 17, 2015

(30) Foreign Application Priority Data
Jun. 21, 2013 (DE) 10 2013 211 837.6

(51) Int. Cl. A61B 5/145; A61B 5/1459; A61B 5/00; A61B 5/1473

(52) U.S. Cl. CPC A61B 5/14503; A61B 5/1473; A61B 5/1459; A61B 5/6852

(57) ABSTRACT

The present invention relates to a catheter (1). To provide a device permitting the real-time tracing and detection of sample material (2), the catheter (1) according to the invention comprises at least one detection device (4, 4a) for supplying real-time detection of the sample material (2), wherein the at least one detection device (4, 4a) has a functionalized surface (5, 5a) for accumulating the sample material (2), a signal converter (6), which converts the accumulation of sample material (2) on the functionalized surface (5, 5a) into a binding signal (7), and a signal line (8) for transmitting the binding signal (7).

The present invention relates to a catheter. Catheters are tubes or pipes of varying diameters for probing, draining, filling or rinsing hollow organs such as the bladder, the stomach, the intestine and blood vessels but also the ear or the heart. Catheters are used in operations, and patients, in intensive care for example, are catheterized in order to supply them with, for instance, vital medicine or, in the case of a balloon catheter, to keep the heart vessels open.

With catheterized patients, particularly with patients in intensive care, there is the risk that pathogens may penetrate the body. Particularly dangerous are infections that may cause a sepsis which may frequently lead on to a life-threatening disruption of the vital function and failure of one or more organs and ultimately to the death of the patient. The intensive care can bridge critical phases by a temporary application or support of the organ functions. However, this is only possible when the occurrence of the infection is detected at an early stage. Once symptoms of a sepsis are already visible externally, such as a raised temperature, heart frequency or breathing rate, it may already be too late for a successful treatment.

Thus, the present invention is based on the object to provide a device which facilitates tracing and detecting sample material so to speak in real-time.

The present invention solves this object by providing a catheter with at least one detection device for the real-time detection of a sample material wherein the at least one detection device comprises a functionalized surface for the accumulation of the sample material, a signal converter which converts the accumulation of the sample material on the functionalized surface into a binding signal and a signal line for transmitting the binding signal. The combination of a catheter and a sensor according to the invention allows for a real-time detection of the sample material also in vivo. A patient may be catheterized, i.e. inserted a catheter according to the invention just like any conventional catheter and fulfill its proper function. Since the catheter according to the invention comprises at least one detection device for the real-time detection of a sample material it is not necessary to wait for externally visible symptoms of an infection, a sepsis in particular, before a sepsis is indicated. Rather, the at least one detection device continuously detects the presence of a specific sample material, for example, an infectious germ. As soon as the sample material is present it will be accumulated on the functionalized surface of the detection device of the catheter according to the invention. The accumulation is instantly transformed into a binding signal by the signal converter which is then transmitted via the signal line and which can then be read out outside of the catheter as a signal indicative of the presence of the predetermined sample material. Thus, there is no delay and it can be recognized at an early stage whether harmful sample material, for example infectious germs, exist in the bloodstream of intensive care patients or in the catheter itself.

The present invention may be further improved by a number of independent further developments each advantageous per se and freely combinable with one another, as described in the following.

According to a first advantageous embodiment, the functionalized surface may be arranged on an external side and/or inner side of the catheter. The placement of the functionalized surface on the external side of the catheter facilitates detecting sample material of the area surrounding a catheter that has been set. In the case of a vein catheter or a cardiac catheter, for instance, sample material from the bloodstream can be detected in real-time by means of the catheter according to the invention. By means of the arrangement of the functionalized surface on an inner side of the catheter it is possible to detect sample material within the fluid stream inside the catheter. Thus, for example, it can be immediately recognized whether an infection or a contamination exists inside the catheter and whether the catheter needs to be replaced. Contaminations of the fluid administered or extracted through the catheter can be identified this way, too.

According to another advantageous embodiment, the at least one detection device can be undetachably connected to the catheter thus facilitating handling of the catheter with the detection device. The at least one detection device may preferably be formed integrally with the catheter. Such an integrally formed embodiment can be obtained, for example, by integrating the at least one detection device into the catheter. An integration can be accomplished in such a way that merely the functionalized surface on an external side and/or inner side of the catheter is exposed such that it interacts with the external surrounding area or, respectively, the internal space of the catheter and that sample material can accumulate on the functionalized surface.

The catheter according to the invention may also comprise a detection device having several functionalized surfaces. For instance, a first functionalized surface at an external side of the catheter and a further, second functionalized surface at an inner side of the catheter may be arranged in such a way that an interaction of the functionalized surface with the fluid flowing inside the catheter as well as with the external surrounding area of the catheter is feasible.

Alternatively, the catheter may comprise more than one detection device comprising a functionalized surface, a signal converter and a signal line wherein, for example, the one detection device is arranged at an external side of the catheter and a second detection device is arranged on an inner side of the catheter.

With an embodiment of the catheter according to the invention having more than one detection device it is also possible to detect different sample materials. Thus, not only information as to whether an infection exists can be retrieved. Rather, a narrower classification of the infectious germ, for instance, a determination of whether the pathogen is gram-negative or gram-positive, can be made.

The catheter according to the invention is suitable for any application which suits conventional catheters. For instance, conceivable is the use as a vein catheter, in urology as a bladder catheter, ureter catheter or nephrostomy catheter, as vessel catheter, balloon catheter or stent catheter in conventional angiography, as cardiac catheter, port catheter, epidural catheter, tube catheter or as a catheter applied in dialysis treatment, e.g. Shaldon catheter, Demers catheter or peritoneal catheter. The catheter according to the invention is also suitable for any catheterization technique and may, for instance, be a disposable catheter and especially a permanent or indwelling catheter as inserted prophylactically in the course of operations, patient monitoring and/or intensive care measures.

The catheter according to the invention may have arbitrary diameters and may be made from materials of the most different kind, for instance, from plastic, rubber, silicone, metal or also glass, with steel and plastic catheters being particularly suitable for cost and sterility reasons.

According to another embodiment, the functionalized surface may be loaded with detection molecules at least in sections. Detection molecules are molecules that specifically bind with the sample material. In terms of the present invention, specific binding means a binding having an affinity high enough to have an association constant (also called binding constant) of at least 10⁴ mol⁻¹, preferably 10⁵ mol⁻¹ and especially 10⁶ mol⁻¹.

According to another embodiment, antibodies, specifically binding fragments of antibodies, antigens, peptides, proteins, nucleic acids, inhibitors, enzymes, endotoxins, enzyme substrates, cofactors of an enzyme, ligands, receptors, chelates, especially metal ion chelates, or other molecules which specifically bind with the sample material, i.e. which bind with a specific affinity, can preferably be used as the detection molecules.

With the catheter according to the invention, sample material of particular target molecules and/or target cells of the most different kind can be detected in real-time. The sample material may be, for instance, a particular membrane structure or a surface protein of a particular pathogen or a disease specific or pathogen specific material or, respectively, a material formed by a pathogen that das not normally occur in the fluid flowing through the catheter or, respectively, in the tissue surrounding the catheter.

The detection molecules can especially bind pathogen specific and/or pathogen associated sample material. This includes special antigens or structures on the surfaces of pathogens but also detectable structures of nucleic acids or material secreted into the surrounding area by the pathogens. The detection molecules can especially bind infection specific and/or infection associated sample material, preferably sepsis specific and/or sepsis associated sample material. A detection molecule may specifically bind O—, H— and pili-antigens or core polysaccharides of the cell membranes, for instance, which facilitates the detection of infectious germs. Endotoxins produced by gram-negative pathogens, e.g. lipid A, or the clumping factor A also constitute a possible pathogen specific sample material.

According to one embodiment, the detection molecules specifically bind with so-called quorum sensing molecules. The quorum sensing molecules, e.g. homoserine lactone, such as homoserine lactone (HSL) 1 to 4 (HSL1: N-(11-carboxy-3-oxoundecanoyl)-L-homoserine lactone; HSL2: N-(5-carboxypentanoyl)-L-homoserine lactone; HSL3: N-(11-carboxy-3-hydroxyundecanoyl)-L-homoserine lactone; HSL4: N-(9-carboxynonanoyl)-L-homoserine lactone); or quorum sensing oligopeptide, that are summarized as autoinducer peptides, serve the chemical communication of unicellular organisms. It has been surprisingly found that an increased concentration of the quorum sensing molecules strongly indicates a sepsis risk so that quorum sensing molecules are a sepsis specific sample material. A catheter according to the invention equipped with a functionalized surface for the accumulation of quorum sensing molecules may thus indicate a suspected sepsis fast and reliably. It is an advantage of detection molecules specifically binding with quorum sensing molecules that not only can the presence of an infection be detected in real-time but also a statement can be made with respect to the gram status of the pathogens because the homoserine lactones are produced only by gram-negative pathogens and the autoinducer peptides are produced only by gram-positive pathogens. Alternatively, sepsis associated sample material can also be detected in real-time by means of the catheter according to the invention. Surprisingly it has further been found that the activity of specific enzymes is changed by the increased concentration of quorum sensing molecules as in the case of an infection. A functionalized surface with detection molecules detecting a change of the activity of such sepsis associated enzymes also allows for the detection of a sepsis in real-time by means of the catheter according to the invention. The increased concentration of quorum sensing molecules in the event of a sepsis results, for example, in an increased activity of the enzymes beta-galaktosidase, beta-hexosaminidase and arylsulfatase A and in a decreased enzyme activity of the enzyme paraoxonase 1.

According to a further embodiment of the catheter of the invention, the signal converter and/or catheter may be coated at least sectionally with a polymer, preferably with a biocompatible polymer. Polymer-coated surfaces are well applicable for the modification of surfaces of the catheter and the signal converter, respectively, due to their versatile properties, and the variety of different polymers and modification possibilities of these polymers facilitate a polymer coating specifically for the respective intended purpose. With the catheter according to the invention, therefore, it is suitable to employ for the coating a protein and/or cell repellent polymer in order to eliminate unwanted deposits on the catheter according to the invention. Suitable are hydrophobic polymers and copolymers such as, e.g. polyethylene glycol, polystyrene or their derivatives as well as hydrophilic polymers such as, e.g. polyacrylates and polyamides as well as natural polymers such as, e.g. polylysine or polysaccharides such as alginate and chitosan.

Suitable for the catheter according to the invention are polymers having functional groups. Via these functional groups the detection molecules can be bonded directly covalently. It is also possible to couple the detection molecules via coupling molecules (so-called linkers) with the desired site and to shape it into a structured functionalized surface. Alginate is an example of a functionalized natural polymer which is protein and/or cell repellent. The coating of a biocompatible polymer may preferably fulfill one of the following requirements:
The biocompatible polymer is formed as a continuous layer. Thus, the entire surface of the catheter may be covered or shielded by the polymer layer. The thickness of the polymer layer preferably is within the range of 0.1 to 10 μm, more preferably within the range of 0.5 to 5 μm and especially preferably within the range of 1 to 2 μm.
The biocompatible polymer has a three-dimensional, preferably filamentous and/or porous structure. The biocompatible polymer has a carbonaceous, branched molecular structure. These structures are excellently suited for the binding of the detection molecules and enlarge the effective binding surface. Within the range of the boundary layer on a surface occupied with this molecular structure the flow of a sample fluid is considerably slowed down. Thus the accumulation of the ligands is facilitated. Possible advantageous embodiments of the structured functional surface are a functional surface having ridges, dents and/or ramifications, and/or a functional surface comprising at least partially a spiral, coiled, volute, wavelike, helical, filamentous, brush-like, comb-like, netted, porous, sponge-like structure.
The biocompatible polymer is preferably operatively connected to the carrier, for example, the catheter or signal converter, via functional groups preferably by chemical binding, especially preferably by a covalent bond.
The biocompatible polymer is a hydrogel.
The biocompatible polymer comprises saturated groups of atoms and covalently bonded detection receptors in order to prevent unwanted interactions with blood components and the binding of nonspecific cells and molecules.
The biocompatible polymer is cross-linked.
The biocompatible polymer comprises the functional surface or constitutes it. The functional surface is preferably located at the surface of the biocompatible polymer. The biocompatible polymer may be directly loaded with the detection molecules.
The biocompatible polymer may form a matrix that prevents the binding of nonspecific cells or interactions with body fluids.

In a further advantageous embodiment of the invention the functionalized surface and/or the polymer may be coated with a protective layer protecting the functionalized surface, its detection molecules in particular, and/or the biopolymer against external factors occurring with sterilization. The protective layer may preferably fulfill one of the following requirements:
The protective layer is soluble in fluids, body fluids in particular, preferably in blood. Thus, the functional surface can be automatically uncovered as soon as the protective layer comes into contact with the sample fluid.
The protective layer is biocompatible. Thus, defense actions of the body during an in vivo application of the detection device are extensively avoided.
The protective layer is organic crystalline. The protective layer comprises at least one of the following components: alginates, preferably highly purified alginates, polyethylene glycols, cyclic and non-cyclic oligosaccharides, polysaccharides, antioxidant amino acids, proteins or vitamins. Such components are biocompatible and easily soluble.

According to a further embodiment, the detection molecules may be coupled with the signal converter or a polymer coating the signal converter directly via a covalent bond or indirectly via a coupling molecule. Thus a reliable and secure connection of the functionalized surface with the signal converter is guaranteed.

According to a further embodiment, the at least one detection device of the catheter according to the invention can comprise an electrochemical signal converter, an optical signal converter, an acoustical signal converter, an electrical signal converter, a thermal signal converter and/or a piezo-electric signal converter. Well suited are electrochemical and/or optical signal converters which are robust and which allow for a reliable conversion of the accumulation of the sample material on the functionalized surface into a binding signal. The electrochemical signal converter may, for instance, convert the binding into a change of the resistance, of the impedance or of the current flow. An optical signal converter may output as a binding signal a change of the light refraction as occurring with surface plasmon resonance spectroscopy.

According to one embodiment, the signal converter may comprise at least one electrode or at least one prism or at least one optical fiber section. The optical fiber section may have an optical fiber core coated with a metal layer. The metal layer may be coupled with the functionalized surface.

At the electrode, the change of the resistance or of the impedance, respectively, may be picked up and put out as binding signal. At the prism or at the optical fiber section, the change of the light refraction may be output as binding signal.

According to one embodiment, the catheter according to the invention comprises detection molecules and/or a functionalized surface that change structurally on binding of the sample material, their structural change effecting a change of the current flow or entailing a change of the light refraction, respectively. Such detection molecules which are modifiable substances may be immobilized on a carrier, directly on the signal converter or on a coating of the signal converter via a linker system, for example.

In order to convey to the outside the binding signal output by the signal converter which is specific to the accumulation of the sample material and in order to detect the accumulation of the sample material in real-time, the catheter according to the invention comprises a signal line. The signal line may be, for instance, an electrical conductor conveying a change of the current flow or of the resistance/the impedance, respectively. In another embodiment, the signal line may be an optical fiber conveying light to the signal converter and again conveying for example a change of the light refraction as a binding signal away from the signal converter to the outside.

According to one embodiment, the signal line may be arranged at the catheter and/or may be enclosed in the catheter. With this embodiment the catheter itself acts as carrier material for the signal line. Since meanwhile especially electrical conductors such as metallic wires and optical fibers, e.g. glass fibers, are very flexible and producible with small diameters, arranging or integrating the signal line at or inside the catheter, respectively, is easily possible. For instance, the signal line may be co-extruded together with the catheter or introduced, interlaced or molded into the catheter.

For the detection device to be less susceptible to interfering signals and background noise and in order to improve an isolation of the actual binding signal, the at least one detection device of the catheter according to the invention may comprise a shield blocking interfering signals occurring, for example, when the catheter is used near the heart. It is another possibility to provide the at least one detection device with at least one reference measuring device. The reference measuring device picks up the background noise or the interfering signal, respectively, and also permits filtering out of the disruptive factors and the background noise, respectively, thus permitting a reliable statement as to whether an actual accumulation of the sample material has occurred at the functionalized surface.

Hereinafter, the invention is explained by way of example by means of drawings. The combinations of features explained by reference to the drawings may be changed, however, according to the aforementioned explanations. Thus, for example, individual features of the embodied catheters with detection device can be waived if these features do not offer a substantial advantage for a specific application. Conversely, one of the above described features may be added if the advantage related to this feature is required for the respective application.

The drawings show:

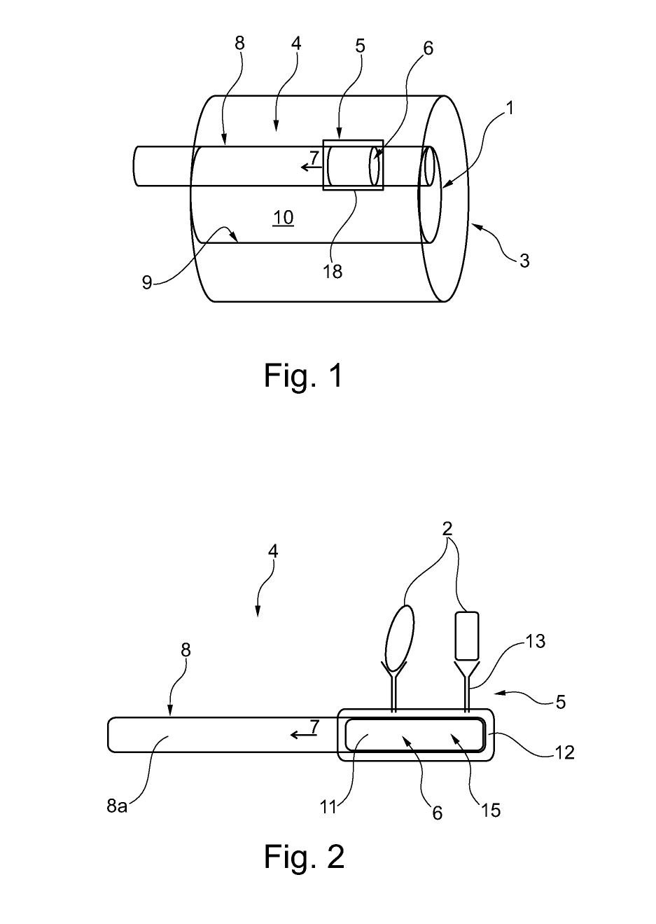

FIG. 1 a schematic perspective illustration of a first embodiment of the catheter according to the invention placed in a lumen;

FIG. 2 a schematic illustration of a detection device;

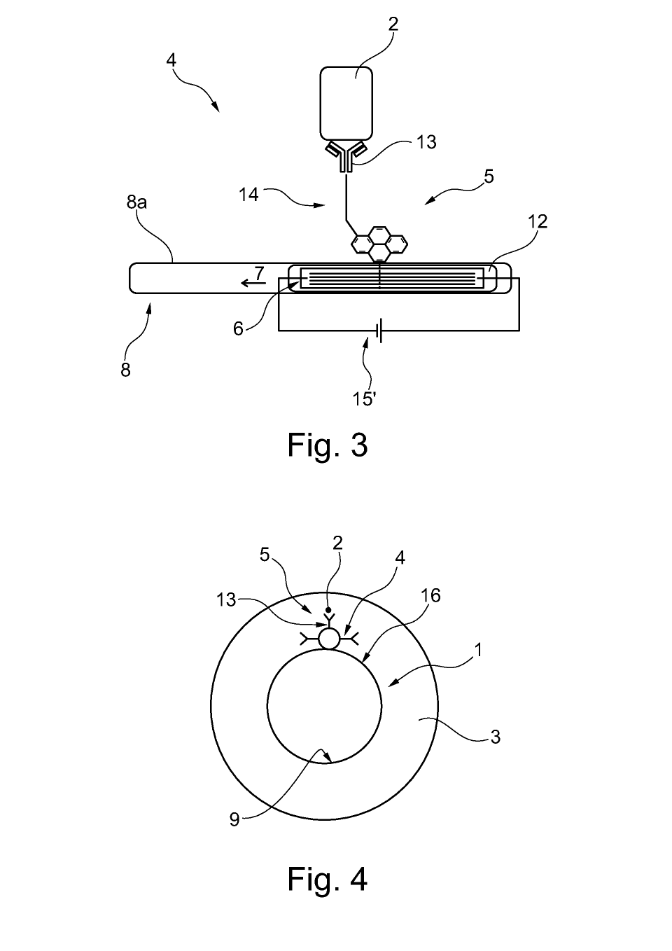

FIG. 3 a schematic illustration of a detection device according to an alternative embodiment;

FIG. 4 a cross section of a catheter according to the invention placed in a lumen according to a second embodiment;

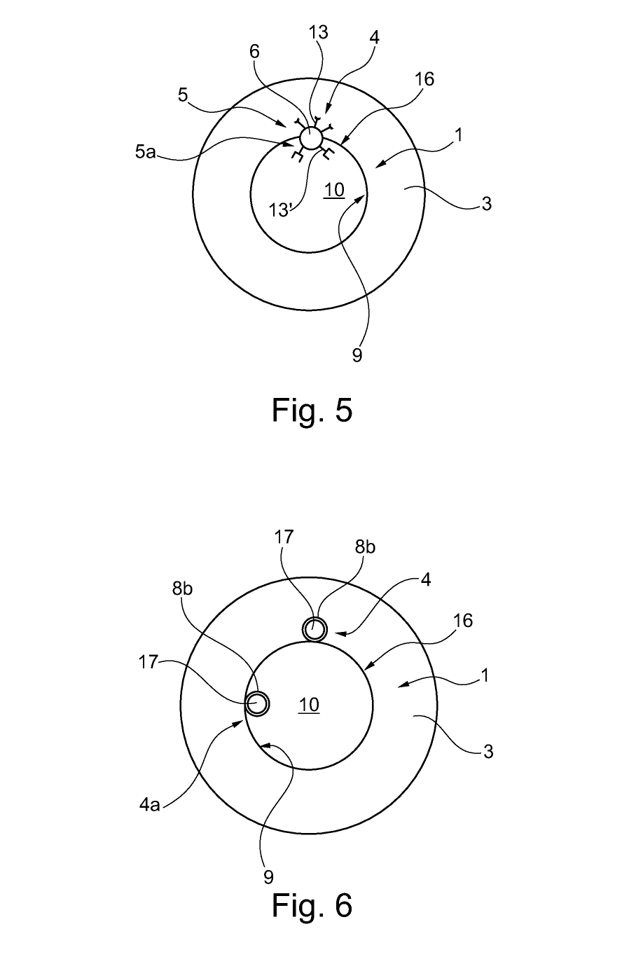

FIG. 5 a cross section of a catheter according to the invention placed in a lumen according to a third embodiment;

FIG. 6 a cross section of a catheter according to the invention placed in a lumen according to a fourth embodiment;

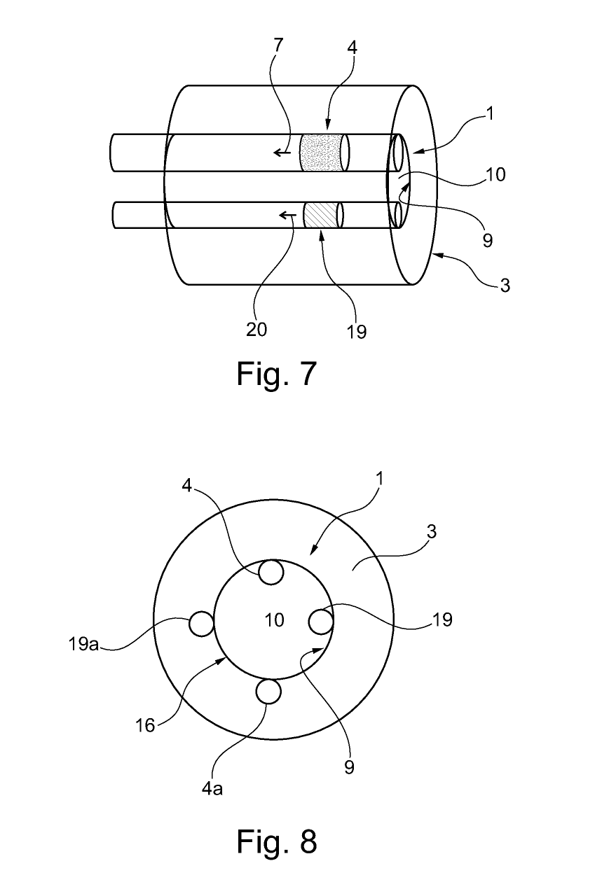

FIG. 7 a schematic perspective embodiment of a catheter according to the invention according to a fifth embodiment;

FIG. 8 a cross section of a catheter according to the invention placed in a lumen according to a sixth embodiment.

The catheter 1 according to the invention and its individual components are explained in detail below with reference to the enclosed drawings.

The catheter 1 according to the invention allows for in vivo and intravascular detection of the binding of a sample material 2 in real-time without the need for removing the catheter 1 from the body.

In the following, a first embodiment of the catheter according to the invention is explained in more detail with reference to FIG. 1. In FIG. 1 it is shown how the catheter 1 according to the invention is placed in a lumen 3 which is exemplified and which can be a blood vessel, for instance. The drawings generally depict the indications of size of the catheter 1, the lumen 3 as well as the detection device 4 of the catheter merely schematically and not true to scale.

The catheter 1 according to the invention comprises a detection device 4 for the real-time detection of the sample material 2, for example, specific pathogenic cells such as Staphylococcus aureus or other infection bacteria or infectious fungi. The detection device 4 comprises a functionalized surface 5 for the accumulation of the sample material 2. The functionalized surface will be explained in more detail below by reference to FIGS. 2 and 3. The detection device 4 further comprises a signal converter 6 converting the accumulation of the sample material 2 on the functionalized surface 5 into a binding signal 7.

Furthermore, the detection device 4 of the catheter 1 according to the invention comprises a signal line 8 for transmitting the binding signal 7, as indicated by an arrow in FIG. 1 by way of example. Via the signal line 8 which can be an electrical conductor 8a or an optical conductor 8b, for example, the binding signal 7 is conveyed away from the detection device 4 and can emit the binding of the sample material 2 on the functionalized surface 5 of the catheter 1 according to the invention outside of the body lumen 3 in real-time. This facilitates recognizing infectious germs, for example, in the bloodstream already at a very early stage which can provide a vital advantage in time especially in case of a sepsis for taking appropriate life-saving counteractive measures in good time.

In the embodiment shown in FIG. 1, the functionalized surface 5 is positioned on the inner side 9 of the catheter. This configuration on the inner side 9 of the catheter 1 allows detecting infections or other undesirable sample material in the internal space 10 of the catheter in real-time and corresponding precautionary measures to be taken, for instance, exchanging the contagious catheter, so the infection does not penetrate the body through the catheter.

With the catheter 1 according to the invention shown in FIG. 1, the detection device 4 is undetachably connected to the catheter 1 by forming the detection device 4 integrally with the catheter 1. For this purpose the detection device is molded with the inner side 9 of the catheter 1 or embedded into it, respectively.

In the following, by reference to FIGS. 2 and 3 two exemplary embodiments of a detection device 4 which can be employed in the catheter 1 according to the invention are explained in more detail.

In FIG. 2 a first embodiment of a detection device 4 is shown. The detection device 4 comprises a functionalized surface 5 for the accumulation of the sample material 2, a signal converter 6 and a signal line 8. In the embodiment shown, the signal line 8 is an electrical conductor 8a which can transmit an electrical binding signal 7 produced by the signal converter 6. In the embodiment shown, the electrical conductor 8a is the guide wire which at the same time is used for introducing the catheter 1 into the respective body lumen 3. For reasons of clarity, the depiction of the lumen 3 as well as of the catheter coating has been omitted.

In the shown embodiment, the signal converter 6 is an electrochemical signal converter that is composed of a gold-coated surface 11 of the electrical conductor 8a. The gold-coated surface 11 forms an electrode 15.

In the embodiment shown in FIG. 2, the functionalized surface 5 comprises detection molecules 13 which in the shown embodiment are produced by antigens against infectious fungi and bacteria, respectively, as sample material 2. The detection molecules 13 are coupled to the signal converter 6. In the embodiment shown, a polymer 12 is envisaged for coupling. The polymer 12 coats the signal converter 6 so that, on the one hand, it is protected against outside influences and; on the other hand, non-specific and undesirable interactions of the signal converter 6 with the sample material 2 are excluded. In the embodiment shown in FIG. 2, a functionalized hydrogel, e.g. a functionalized alginate gel, represents the polymer 12 coating the signal converter 6. The detection molecules 13 of the functionalized surface 5 are coupled with the polymer 12. For coupling the detection molecules 13 with the polymer 12, antibodies which form the detection molecules 13 may be bonded to the functional groups of the alginate. Binding may occur, for instance, by directly chemically binding the antibody to the functional groups of the alginate via the formation of a covalent bond. Alternatively, as shown in FIG. 3 and explained in more detail below, a coupling molecule 14, also called linker, may be used which is bonded to the polymer 12, on the one hand, and to the detection molecules 13, on the other hand.

If sample material 2 that is to be detected with the catheter 1 according to the invention is present, this sample material 2 will bind to detection molecules 13 of the detection device 4 which are specific to it. In the gold layer 11 representing an electrode 15 of the signal converter 6, the binding of the sample material 2 to the antibodies is transformed into a binding signal 7. In the shown embodiment, the transformation occurs due to the binding of the sample material 2 to the antibodies as detection molecules 13 resulting in a change of the current flow and eventually of the resistance in the electrode 15 formed by the gold layer 11. This resistance change is subsequently conveyed away as a binding signal 7 via the electrical line 8a and indicates outside of the lumen 3 in real-time that a binding of the sample material 2 exists.

In FIG. 3, an alternative embodiment of the detection device 4 of FIG. 2 is illustrated. Hereinafter, only the differences between the detection device 4 of FIG. 3 and the detection device 4 of FIG. 2 are dealt with. Identical reference signs are used for elements having a function and/or structure identical to the elements of the previous figures.

Like with the detection device 4 of FIG. 2, the detection device 4 of FIG. 3 comprises an electrical conductor 8a as signal line 8, a signal converter 6 coated with a polymer 12 and a functionalized surface 5 comprising antibodies as detection molecules 13. In the embodiment of FIG. 3, the antibody 13 is not directly bonded to the polymer 12 but via a coupling molecule 14.

Small molecules having, for example, two identical (homobifunctional) or two different (heterobifunktonal) functional groups are designated linkers. Likewise the length of the linker is relevant to the function. Zero-length crosslinkers are used for a bonding of two molecules without a spacer. The use of a linker, especially with complex molecules like enzymes or antibodies, may have a promoting effect on the biological activity of the immobilized structure. By means of the linker, the active centre or the active domain of the molecule is conveyed further away from the core structure at which the molecule is immobilized. Thus the risk of an inactivation by the immobilization is reduced. Another possibility is to choose the linker such that it binds with only one specific structure in the target molecule thus leaving intact the active region of the molecule. For the coupling of an IgG antibody to carboxyl groups in the polymer, the zero-length crosslinker EDC (1-ethyl-3-[3-dimethylaminopropyl] carbodiimide hydrochloride) can be used which catalyzes the formation of a peptide bond between a primary amino group in the antibody and a carboxyl group of the polymer.

In the embodiment shown in FIG. 3, the signal converter 6 comprises an electrode array 15′ that detects a change in resistance that is provoked by the binding of the sample material 2 to the antibody 13 and an associated structural change of the coupling molecule 14. The resistance change is emitted by the signal converter 6 as a binding signal 7 and transported to the outside via the electrical conductor 8a as signal line 8. The binding of the sample material 2 to the antibodies thus effects a modification of the functionalized surface 5 or the coupling molecules 14, respectively, which is reflected in a change in current flow that can be output as a change in resistance or impedance, respectively, as binding signal 7.

In the following, by reference to FIG. 4 a second embodiment of the catheter 1 according to the invention is explained in more detail. In FIG. 2, the catheter 1 according to the invention placed in a lumen 3 is represented by a cross-sectional view. Below, only the differences between the catheter 1 of the second embodiment and the catheter of the first embodiment as shown in FIG. 1 are dealt with.

With catheter 1 of the second embodiment, the detection device 4 is not placed on the inner side 9 but on the external side 16 of the catheter. Thus the functionalized surface 5 of the detection device 4 is arranged in the lumen 3. Thereby sample material 2 from the lumen 3, such as a blood vessel, can be detected in real-time. For example, infectious pathogens associated with sepsis can be detected with the catheter 1 according to the invention at a very early stage and in real-time without having to take a sample or previously removing the catheter 1 from the lumen 3.

Subsequently a third embodiment of a catheter 1 according to the invention as illustrated in FIG. 5 will be explained.

FIG. 5 shows a cross section of a catheter 1 according to the invention placed in a lumen 3 which essentially corresponds to the illustration in FIG. 4.

The catheter 1 of the invention according to the third embodiment is characterized by the detection device 4 having a first functionalized surface 5 arranged on the external side of the catheter 1 as well as a second functionalized surface 5a that is arranged on the inner side 9 of the catheter 1.

With catheter 1 of the third embodiment, the detection device 4 is integrated into the catheter 1. For example, the detection device 4 may be molded into the catheter body in such a way that only the first functionalized surface 5 is exposed and readily accessible on the external side 16 and only the second functionalized surface 5a is exposed and readily accessible on the inner side 9 of the catheter 1. Thus it can be detected whether sample material 2 exists in the lumen 3 and/or in the internal space 10 of the catheter.

In the third embodiment of the catheter 1 according to the invention as shown in FIG. 5, the functionalized surface 5 comprises detection molecules 13 which are different from the detection molecules 13′ of the second functionalized surface 5a. Thus different sample materials 2 can be detected. If the first functionalized surface 5 and the second functionalized surface 5a are coupled with the signal converter 6 in such a way that different binding signals 7 are emitted depending on whether binding of the sample material 2 to the first functionalized surface 5, to the second functionalized surface 5a or to both functionalized surfaces 5 and 5a occurs, a statement can be made about where the sample material binds, i.e. whether, for instance, an infection exists in the lumen, in the catheter or in the lumen and in the catheter.

Below, by reference to FIG. 6 a fourth embodiment of the catheter 1 according to the invention is explained. The catheter 1 according to the fourth embodiment is also capable of detecting the accumulation of sample material 2 in the internal space 10 of the catheter as well as in the lumen 3.

In contrast to the catheter of the third embodiment of FIG. 5, the catheter 1 of the fourth embodiment according to FIG. 6 comprises two detection devices 4 and 4a. The first detection device 4 is arranged at the external side 16 of the catheter and can thus detect the presence of sample material 2 in the lumen 3. The second detection device 4a is arranged on the inner side 9 of the catheter 1 and can thus detect the presence of sample material 2 that specifically binds to detection molecules of the second detection device 4a in the internal space 10 of the catheter.

The detection devices 4, 4a of the fourth embodiment of FIG. 6 are optical detection devices employing the measuring principle of the surface plasmon resonance. For that purpose the detection devices 4, 4a of the fourth embodiment comprise an optical conductor 8b, e.g. a fiber optic cable, having a metal coated optical fiber section 17 as the optical signal converter 6. Through the optical fiber 8b polarized light is fed in total internal reflection and proceeds to the metal coated optical fiber section 17 having arranged thereon the functionalized surface 5. If no sample material 2 binds to the functionalized surface, the angular spectrum of the totally reflected polarized light at a particular angle will display a minimum. If, on the other hand, sample material 2 binds to the functionalized surface 5, this will affect the refractive index of the analyte and thus result in an angular displacement that can be emitted as binding signal 7 via the metal coated optical fiber section 17 as signal converter 6 of the optical detection device and via the optical conductor 8b. Alternatively, a prism of a surface plasmon resonance detector can be used as optical signal converter 6.

With the design of the catheter 1 according to the invention of the fourth embodiment of FIG. 6 having two detection devices 4, 4a, one being arranged on the external side 16 and the other one on the inner side 9 of the catheter 1, it is possible to detect also with this embodiment whether sample material 2 is present in the internal space 10 of the catheter 1 and/or in the lumen 3.

Below, by reference to FIG. 7 a fifth embodiment of a catheter 1 according to the invention is illustrated. The illustration of FIG. 7 essentially corresponds to FIG. 1 so that in the following merely the differences between the catheter 1 of the first embodiment of FIG. 1 and of the fifth embodiment of FIG. 7 are dealt with.

With the catheter of FIG. 1, a shield 18 is provided insulating the detection device 4, more precisely the signal converter 6 together with the functionalized surface 5 against external disruptive factors. By means of the shield 18, a reduction of external factors is achieved which could undesirably affect the binding of the sample material 2 and, respectively, distort the conversion of the binding by the signal converter 6 into the binding signal 7.

The catheter 1 according to the fifth embodiment of FIG. 7 comprises no shield 18 but instead a reference measuring device 19. The reference measuring device 19 is also arranged on the inner side 9 of the catheter 1 and facilitates a reference measurement representative of a background noise. By subtracting from the binding signal 7 the background signal 20 emitted by the reference measuring device 19, the signal effectively characteristic of the binding of the sample material 2 can be isolated.

The reference measuring device 19 may be designed identically to the detection device 4, for instance, with the only difference that the functionalized surface 5 comprises either no detection molecules 13 or only reference molecules that do not bind the sample material 2 to be detected.

Finally, in FIG. 8 a catheter 1 of the invention according to a sixth embodiment is shown. FIG. 8 again shows a schematic cross section of a catheter 1 according to the invention placed in a lumen 3 such as analogously illustrated, for example, in FIG. 4 for the catheter 1 of the second embodiment.

The sixth embodiment of the catheter 1 according to the invention is a modification of the fifth embodiment of FIG. 7. Also with the sixth embodiment of FIG. 8, an electrochemical detection device 4 as well as a reference device 19 for filtering the background noise is arranged on the inner side 9 of the catheter 1.

Moreover, the catheter 1 according to the sixth embodiment of FIG. 8 comprises a further electrochemical detection device 4a on the external side 16 of the catheter 1 and a further reference measuring device 19a also placed on the external side 16 of the catheter 1. Thus, a binding signal 7 displaying the binding of sample material 2 in the catheter filtered from the background noise can be emitted not only from the internal space 10. Additionally, sample material can also be detected in the lumen 3 wherein the binding signal 7 is also isolated and reduced by the background noise.

### LIST OF REFERENCE SIGNS

1 catheter
2 sample material
3 lumen
4, 4a detection device
5, 5a functionalized surface
6 signal converter
7 binding signal
8 signal line
8a electrical conductor
8b optical conductor
9 inner side of the catheter
10 internal space of the catheter
11 gold coating
12 polymer
13, 13′ detection molecule
14 coupling molecule
15, 15′ electrode/electrode array
16 external side of the catheter
17 optical fiber section
18 shield
19, 19a reference measuring device
20 background signal

1. A catheter comprising at least one detection device for the real-time detection of a sample material wherein the at least one detection device comprises a functionalized surface for the accumulation of the sample material, a signal converter which converts the accumulation of the sample material on the functionalized surface into a binding signal and a signal line for transmitting the binding signal.

2. The catheter according to claim 1 wherein the functionalized surface is arranged on the external side and/or the inner side of the catheter.

3. The catheter according to claim 1 wherein the at least one detection device is undetachably connected to the catheter and further wherein the at least one detection device is formed integrally with the catheter.

4. The catheter according to claim 1 wherein the functionalized surface is at least sectionally loaded with detection molecules.

5. The catheter according to claim 4 wherein the detection molecules comprise antibodies, binding fragments of antibodies, antigens, peptides, proteins, nucleic acids, ligands, receptors, chelates, haptens, enzymes, enzyme inhibitors, enzyme substrates, cofactors of enzymes, endotoxins or other molecules which specifically bind with the sample material arc used as the detection molecules.

6. The catheter according to claim 1 wherein the detection molecules bind with pathogen specific and/or pathogen associated sample material.

7. The catheter according to claim 1 wherein at least one of the detection molecules and the functionalized surface structurally changes upon binding of the sample material.

8. The catheter according to claim 1 wherein the signal converter and/or the catheter is at least sectionally coated with a polymer.

9. The catheter according to claim 8 wherein the polymer has functional groups.

10. The catheter according to claim 1 wherein the detection molecules are couple directly via a covalent bond or indirectly via a coupling molecule to the signal converter or a polymer coating the signal converter.

11. The catheter according to claim 1 wherein the at least one detection device further comprises an electrochemical, an optical, an acoustical, an electrical, a thermal and/or a piezo-electric signal converter.

12. The catheter according claim 11 wherein the signal converter further comprises at least one electrode or an optical fiber section.

13. The catheter according to claim 1 wherein the signal line is an electrical conductor or an optical fiber.

14. The catheter according to claim 1 wherein the signal line is arranged at the catheter and/or is enclosed in the catheter.

15. The catheter according to claim 1 wherein the at least one detection device further comprises a shield against disruptive factors and/or at least a reference measuring device.

16. The catheter according to claim 3 wherein the at least one detection device is undetachably connected to the catheter, and further wherein the at least one detection device is integrated into the catheter.

17. The catheter according to claim 6 wherein the pathogen specific and/or pathogen associated sample material is an infection specific and/or infection associated sample material.

18. The catheter according to claim 6 wherein the pathogen specific and/or pathogen associated sample material is a sepsis specific and/or sepsis associated sample material.

19. The catheter according to claim 8 wherein the polymer is a biocompatible polymer.

20. The catheter according claim 12 wherein the signal converter comprising an optical fiber section is metal coated.
